SEQUENCE LISTING (4 sequences)

SEQ ID NO: 1 - protein, 124 aa, Homo sapiens
MPACRLGPLAAALLLSLLLFGFTLVSGTGAEKTGVCPELQADQNCTQECVSDSECADNLKCCSAGCATFCSLPNDKEGSCPQVNINFPQLGLCRDQCQVDSQCPGQMKCCRNGCGKVSCVTPNF

SEQ ID NO: 2 - protein, 22 aa, Homo sapiens
LQVQVNLPVSPLPTYPYSFFYP

SEQ ID NO: 3 - protein, 10 aa, Homo sapiens
LLCPNGQLAE

SEQ ID NO: 4 - protein, 28 aa, Homo sapiens
ALFHWHLKTRRLWEISGPRPRRPTWDSS

(19) United States
(12) Patent Application Publication
(10) Pub. No.: US 2014/0315734 A1
(43) Pub. Date: Oct. 23, 2014

(54) METHODS AND COMPOSITIONS FOR ASSIGNING LIKELIHOOD OF ACUTE KIDNEY INJURY PROGRESSION

(71) Applicants: William D. Arnold, San Diego, CA (US); Christelle Jost, San Diego, CA (US); Brian Noland, Temecula, CA (US); Jonathan Gary, San Diego, CA (US); Joseph Buechler, Carlsbad, CA (US); Vance Wong, Cardiff, CA (US); Scott Harold Rongey, San Diego, CA (US); Uday Kumar Veeramallu, San Diego, CA (US); Kelline Marie Rodems, Oceanside, CA (US)

(72) Inventors: William D. Arnold, San Diego, CA (US); Christelle Jost, San Diego, CA (US); Brian Noland, Temecula, CA (US); Jonathan Gary, San Diego, CA (US); Joseph Buechler, Carlsbad, CA (US); Vance Wong, Cardiff, CA (US); Scott Harold Rongey, San Diego, CA (US); Uday Kumar Veeramallu, San Diego, CA (US); Kelline Marie Rodems, Oceanside, CA (US)

(73) Assignee: ALERE SAN DIEGO INC., San Diego, CA (US)

(21) Appl. No.: 14/130,209

(22) PCT Filed: Jul. 3, 2012
(86) PCT No.: PCT/US12/45421

§ 371 Date: Feb. 12, 2014

Related U.S. Application Data
(60) Provisional application No. 61/504,844, filed on Jul. 6, 2011.

(51) Int. Cl. G01N 33/68

(52) U.S. Cl. CPC G01N 33/6893
USPC 506/9; 435/7.92; 435/6.12; 435/287.2

(57) ABSTRACT

The invention encompasses diagnosis and prognosis in the context of heart or renal failure, particularly in subjects who exhibit a normal body fluid level of a natriuretic peptide. The invention also relates to methods of assigning an increased likelihood that a subject having AKI is susceptible to AKI progression. The invention relates in part to assigning a diagnosis of heart and/or renal failure, and/or an outcome risk (e.g., worsening cardiac and/or renal function or a mortality risk) to a subject based, at least in part, on the result(s) obtained from an assay that detects WAP four-disulfide core domain protein 2 performed on a body fluid sample obtained from a subject.

This application claims the benefit of U.S. provisional patent application No. 61/504,844, which was filed on Jul. 6, 2011 and is incorporated herein by reference in its entirety.

### FIELD OF THE INVENTION

The present invention relates to methods, compositions, and kits for diagnosis, prognosis, and monitoring of heart and/or renal failure. The invention also relates to methods, compositions, and kits for assigning an increased likelihood that a subject having acute kidney injury (AKI) is susceptible to AKI progression.

### BACKGROUND OF THE INVENTION

The following discussion of the background of the invention is merely provided to aid the reader in understanding the invention and is not admitted to describe or constitute prior art to the present invention.

### Heat Failure

Congestive heart failure (CHF) is a fatal disease with a 5-year mortality rate that rivals the most deadly malignancies. For example, in the Framingham Heart Study, median survival after the onset of heart failure was 1.7 years in men and 3.2 years in women. Overall, 1-year and 5-year survival rates were 57% and 25% in men and 64% and 38% in women, respectively. Moreover, a person age 40 or older has a one-in-five lifetime chance of developing congestive heart failure. Heart failure typically develops after other conditions have damaged the heart. Coronary artery disease, and in particular myocardial infarction, is the most common form of heart disease and the most common cause of heart failure.

The appropriate treatments given to patients suffering from heart failure are diverse. For example, diuretics are often given to reduce the increased fluid load characteristic of heart failure; Angiotensin-Converting Enzyme (ACE) inhibitors are a class of vasodilator used to lower blood pressure, improve blood flow and decrease the workload on the heart; Angiotensin II Receptor Blockers (ARBs) have many of the same benefits as ACE inhibitors; and Beta blockers may reduce signs and symptoms of heart failure and improve heart function.

In recent years, natriuretic peptide measurement has dramatically changed the diagnosis and management of cardiac diseases, including heart failure and the acute coronary syndromes. In particular, B-type natriuretic peptide (BNP, human precursor Swiss-Prot P16860), various related polypeptides arising from the common precursor proBNP (such as NT-proBNP), and proBNP itself have been used to diagnose heart failure, determine its severity, and estimate prognosis. In addition, BNP and its related polypeptides have been demonstrated to provide diagnostic and prognostic information in unstable angina, non-ST-elevation myocardial infarction, and ST-elevation myocardial infarction.

BNP and its related peptides are correlated with other measures of cardiac status such as New York Heart Association classification. However, many patients with chronic stable or asymptomatic heart failure will have natriuretic peptide levels in the normal diagnostic range (e.g., BNP levels less than about 100 pg/mL; NT-proBNP levels less than about 400 pg/mL). There is a trade-off in selecting diagnostic cutoff levels for these markers, because lowering the cutoff decreases the false-negative rate (i.e., increased sensitivity and fewer missed diagnoses) but increases the false-positive rate (i.e., decreased specificity and more incorrect diagnoses).

### Renal Failure and Kidney Disease

Renal failure or kidney failure (sometimes referred to as renal insufficiency) describes a medical condition in which the kidneys fail to adequately filter toxins and waste products from the blood. The two forms are acute (acute kidney injury) and chronic (chronic kidney disease); a number of other diseases or health problems may cause either form of renal failure to occur.

Renal failure is described as a decrease in the glomerular filtration rate. Biochemically, renal failure is typically detected by an elevated serum creatinine level. Problems frequently encountered in kidney malfunction include abnormal fluid levels in the body, deranged acid levels, abnormal levels of potassium, calcium, phosphate, and (in the longer term) anemia as well as delayed healing in broken bones. Depending on the cause, hematuria (blood loss in the urine) and proteinuria (protein loss in the urine) may occur. Long-term kidney problems have significant repercussions on other diseases, such as cardiovascular disease.

In recent years, chronic kidney disease (CKD) has become recognized as a major public health problem in the U.S. Until the past few years, kidney failure, the last stage of progressive kidney disease, has been the most visible outcome of CKD. The United States Renal Data Services (USRDS) maintains statistics on treatment of patients with kidney failure by dialysis and transplantation, known as end-stage renal disease (ESRD). The incidence of ESRD has doubted in the U.S. since 1990. This trend seems likely to continue, albeit at a lower rate. A much higher prevalence of earlier stages of chronic kidney disease (CKD) has been inferred. Based on data from Third National Health and Nutrition Examination Survey (NHANES III), there are 8,000,000 individuals in the U.S. with significantly decreased kidney function, who have an estimated glomerular filtration rate (GFR) of less than 60 ml/min/1.73 m².

There are even more individuals with manifestations of kidney damage (particularly albuminuria) without & significant decrease in kidney function. At the current incidence rate of about 100,000 new ESRD cases per year, it is evident that most patients with CKD do not progress to ESRD, but likely succumb to cardiovascular disease, which is also the leading cause of mortality of ESRD patients on maintenance dialysis.

There is currently no cure tor chronic kidney disease. The goals of therapy are to: slow the progression of disease; treat underlying causes and contributing factors; treat complications of disease; and replace lost kidney function. Strategies for slowing progression and treating conditions underlying chronic kidney disease include the following: control of blood glucose, control of high blood pressure, and diet.

In end-stage kidney disease, kidney functions can be replaced only by dialysis or by kidney transplantation. The planning for dialysis and transplantation is usually started in Stage 4 of chronic kidney disease. Most patients are candidates for both hemodialysis and peritoneal dialysis.

More recently it has been shown that combining the chronic kidney disease markers of creatinine-based estimated glomerular filtration rate and mine albumin-to-creatinine ratio with the biomarker cystatin C was associated with improved prediction of end-stage kidney disease and all-cause death.

Chronic kidney disease (CKD) is currently defined by five discrete stages, which are based on the creatinine estimated glomerular filtration rate (eGFR) or urine albumin-to-creatinine ratio (ACR). Clinical laboratories are routinely reporting estimated GFR, and electronic medical records often alert clinicians to the presence of CKD on estimated GFR alone, even though because of several factors, serum creatinine may misclassify individuals. Because routine assessment of the ACR is only recommended for persons with diabetes, initial CKD detection in routine practice is primarily limited to serum creatinine testing. Serum, cystatin C, an alternative biomarker of kidney function, is not routinely used in clinical practice.

There are several stages of CKD, defined in the Executive Summary of the CKD Working Group, which are based on the nominal function or performance of the kidney (see American Journal of Kidney Diseases, Vol 39, No 2, Suppl 1 (February) 2002, ppS 17-S31, the contents of which are incorporated herein by reference.

The inventors have identified markers, which can be used for diagnosis and risk stratification of patients having or suspected of having heart and/or renal failure; and further for assigning a risk of CKD progression in a patient diagnosed with CKD.

### BRIEF SUMMARY OF THE INVENTION

The invention encompasses methods, compositions, and kits for diagnosis, prognosis, and determination of treatment regimens in subjects suffering from or being evaluated for heart and/or renal failure. In various aspects, the invention provides methods for assessing risk of worsening heart and/or renal failure; methods for assigning risk of re-hospitalization in the context of heart and/or renal failure; methods for assigning risk of mortality in the context of heart and/or renal failure, methods of monitoring heart and/or renal failure; and various devices and kits adapted to perform such methods.

In a first embodiment, the invention encompasses methods for risk stratification (i.e., assigning an outcome risk) to a subject. These methods comprise performing an assay that detects WAP four-disulfside core domain protein 2 (also known as “WAP4C” and “HE4”; human precursor Swiss-Prot entry Q14508) on a body fluid sample obtained from a subject, thereby providing one or more assay result(s); and assigning an outcome risk based on the assay result(s) obtained.

In certain embodiments, each assay result is compared so a corresponding baseline (i.e., a diagnostic or prognostic “threshold”) level, which is considered indicative of a “positive” or “negative” result. A variety of methods may be used by the skilled artisan to arrive at a desired baseline. In certain preferred embodiments, the baseline assay result is determined from an earlier assay result obtained from the same subject. That is, the change in a biomarker concentration may be observed over time, and an increased concentration provides an indication of the onset of or worsening, heart and/or renal failure in the subject.

In alternative embodiments, the baseline assay result is determined from a population of subjects, In the case of the use of the markers of the invention for diagnosis, the population may contain some subjects which suffer from heart and/or renal failure, and some which do not; in the case of their use for prognosis, the population may contain some subjects which suffer from some outcome (e.g., heart and/or renal mortality; worsening heart and/or renal failure; improving heart and/or renal failure, etc.), and some which do not as described hereinafter, a threshold is selected which provides an acceptable level of specificity and sensitivity in separating the population into a “first” subpopulation exhibiting a particular characteristic (e.g., having an increased risk of worsening heart and/or renal failure) relative to the remaining “second” subpopulation that does not exhibit the characteristic. As discussed herein, a preferred threshold value separates this first and second population by one or more of the following measures of test accuracy;

an odds ratio of at least about 2 or more or about 0.5 or less, more preferably at least about 3 or more or about 0.33 or less, still, more preferably at least about 4 or more or about 0.25 or less, even more preferably at least about 5 or more or about 0.2 or less, and most preferably at least about 10 or more or about 0.1 or less;

at least 75% sensitivity, combined with at least 75% specificity;

a ROC curve area of at least 0.6, more preferably 0.7, still more preferably at least 0.8, ever more preferably at least 0.9, and most preferably at least 0.95; and/or

a positive likelihood ratio (calculated as sensitivity/(1-specificity)) of at least 5, more preferably at least 10, and most preferably at least 20; or a negative likelihood ratio (calculated as (1-sensitivity)/specificity) of less than or equal to 0.3, more preferably less than or equal to 0.2, and most preferably less than or equal to 0.1. The term “about” in this context refers to +/−5% of a given measurement.

The present risk stratification methods preferably assign a “near-term” risk of worsening heart and/or renal failure or cardiovascular mortality. By “near term” is meant within 30 days. As described hereinafter, the methods preferably assign a risk within 7 days, more preferably within 5 days, and still more preferably within 3 days.

Preferred assay methods comprise performing an immunoassay that detects a marker of interest. Antibodies for use in such assays will specifically bind the marker of interest, and may optionally also bind one or more polypeptides that are “related” thereto, as described hereinafter with regard to related markers. Such immunoassays may comprise contacting said body fluid sample with a solid phase comprising antibody that detects the marker, and detecting binding to that antibody, although assay formats that do not require the use of a solid phase are known in the art. While the invention is generally described in terms of immunoassays, other binding entities (e.g., aptamers), which are not based on an immunoglobulin scaffold may be used in lieu of antibodies in such methods. Preferably, the body fluid sample is selected from the group consisting of urine, blood, serum, and plasma.

While use of WAP four-disulfide core domain protein 2 alone is described herein, it is not intended that a prognosis must be assigned based exclusively on the WAP four disulfide core domain protein 2 assay result. Rather, the skilled artisan will understand that a diagnosis, prognosis, monitoring, etc., can also consider numerous additional clinical variables as described hereinafter, provided that the assay results are variables considered during the diagnostic process; that is, the assay result(s) are used to increase or decrease the probability that the subject under study suffers from heart and/or renal failure. As described in additional detail hereinafter, assays that detect various markers (both subject-derived and physical characteristics) may be combined, including assays that detect various natriuretic peptides such as BNP, NT-proBNP, and proBNP; markers related to inflammation such as myeloperoxidase, soluble FLT-1, C-reactive protein, and placental growth factor; markers related to cardiac damage such as cardiac troponins and CK-MB; markers of renal damage such as serum creatinine, creatinine clearance rates, cystatin C, and glomerular filtration rates: and variables such as urine output levels, age, the presence or absence of various cardiovascular risk factors such as diabetes, hypertensions body mass, smoking status; etc.

In still another embodiment, the invention encompasses methods for monitoring heart and/or renal disease, and in particular heart and/or renal failure, in a patient. These methods comprise performing an assay method that is configured to detect an assay that detects WAP four-disulfide core domain protein 2 on serially collected body fluid samples obtained from a subject, thereby providing a plurality of assay results. A worsening heart and/or renal disease status may be assigned to the patient if the assay results are Increasing with time. In the alternative, an improving heart and/or renal disease status may be assigned to the patient if the assay result(s) are decreasing with time.

In certain embodiments, reagents for performing such assays are provided in an assay device, and such assay devices may be included in such a kit. Preferred reagents comprise one or more solid phase antibodies, the solid phase antibody comprising antibody that detects the intended target(s) bound to a solid support. In the case of sandwich immunoassays, such reagents can also include one or more detectably labeled antibodies, the detectably labeled antibody comprising antibody that detects the intended target(s) bound to a detectable label. Additional optional elements that may be provided as part of an assay device are described hereinafter.

In yet another embodiment, the invention encompasses methods of assessing renal function in a subject, comprising performing an assay method that detects WAP four-disulfide core domain protein 2 on a body fluid sample obtained from the subject, thereby providing an assay result; and relating the assay result to the subject or patient's renal function.

In another embodiment, the invention encompasses a method of assessing renal function in a subject suspected of having renal injury comprising: performing an assay that detects an amount of WAP four-disulfide core domain protein 2 in a biological sample obtained from said subject; and correlating the amount of WAP four-disulfide core domain protein 2 with the subject's renal function.

In certain embodiments, the relating or correlating step comprises determining a concentration of WAP four-disulfide core domain protein 2 and relating said concentration to the occurrence or nonoccurrence of acute kidney injury in the subject. In certain embodiments, the relating step comprises assigning an occurrence of acute kidney injury to the subject when said WAP four-disulfide core domain protein 2 concentration is greater than a predetermined baseline or threshold WAP four-disulfide core domain protein 2 concentration, or assigning a nonoccurrence of acute kidney injury to the subject when said WAP four-disulfide core domain protein 2 concentration is less than a predetermined WAP four-disulfide core domain protein 2 baseline concentration. In other embodiments, the predetermined WAP four-disulfide core domain protein 2 baseline concentration is determined by performing an assay method that detects WAP four-disulfide core domain protein 2 on a body fluid sample obtained from said patient at a time earlier than the time at which the body fluid sample used to provide the assay result was obtained. In certain other embodiments, the predetermined WAP four-disulfide core domain protein 2 baseline concentration is determined from a first population of subjects suffering from acute kidney injury and a second population of subjects not suffering from acute kidney injury.

In certain embodiments, the predetermined WAP four-disulfide core domain protein 2 baseline concentration separates said first population from the second population with an odds ratio of at least 2 or more or 0.5 or less.

In certain embodiments, the predetermined WAP four-disulfide core domain protein 2 baseline concentration separates said first population from the second population with an odds ratio of at least 3 or more or 0.33 or less.

In certain embodiments, the predetermined WAP four-disulfide core domain protein 2 baseline concentration separates said first population from the second population with a specificity of at least about 70%.

In certain embodiments, the predetermined WAP four-disulfide core domain protein 2 baseline concentration separates said first population from the second population with a sensitivity of at least about 70%.

In certain embodiments, the body fluid sample includes, but is not limited to, urine, blood, serum, plasma, saliva, stool, etc.

In certain embodiments, the threshold WAP four-disulfide core domain protein 2 concentration is between about 15 ng/mL and about 25 ng/mL. In certain embodiments, the threshold WAP four-disulfide core domain protein 2 concentration is about 20.2 ng/mL.

In other embodiments, the threshold WAP four-disulfide core domain protein 2 concentration is determined by detecting protein levels in said biological sample. In certain embodiments, the protein levels are detecting using ELISA.

In other embodiments, the threshold WAP four-disulfide core domain protein 2 concentration is determined by detecting mRNA encoding WAP four-disulfide core domain protein 2 in said biological sample. In certain embodiments, the mRNA is detected by RT-PCR.

Another embodiment encompasses methods of assessing renal function in a subject comprising:

a. obtaining a biological sample from said subject;
b. determining a concentration of WAP four-disulfide core domain protein 2 in the sample;
c. comparing the concentration of WAP four-disulfide core domain protein 2 in the sample to a threshold concentration of WAP four-disulfide core domain protein 2; and
d. determining if the subject is likely to have renal injury if the concentration of WAP four-disulfide core domain protein 2 in the sample is within a certain threshold concentration.

Another embodiment encompasses a method of determining a threshold WAP four-disulfide core domain protein 2 concentration in a subject in a subject comprising:

a. obtaining a biological sample from said subject;
b. determining a concentration of WAP four-disulfide core domain protein 2 in the sample;
c. comparing the concentration of WAP four-disulfide core domain protein 2 in the sample to a threshold concentration of WAP four-disulfide core domain protein 2; and
d. determining if the subject is likely to have renal injury if the concentration of WAP four-disulfide core domain protein 2 in the sample is within a certain threshold concentration.

In certain embodiments, the method further comprises determining one or more additional variables selected from the group consisting of a BNP level, an NT-proBNP level, a proBNP level, a myeloperoxidase level, a soluble FLT-1 level, a C-reactive protein level, a cardiac troponin level, an NGAL level, a serum creatinine level, a creatinine clearance rate, a cystatin C level, and a glomerular filtration rate for said patient.

In certain embodiments, the method further comprises determining one or more additional variables selected from the group consisting of a urine output level for said patient, age of said patient, the presence or absence of diabetes in said patient, and the presence or absence of hypertension in said patient.

In certain embodiments, the acute kidney marker concentration is between about 15 ng/mL and about 25 ng/mL, or about 1 ng/mL and about 22 ng/mL. In other embodiments, the acute kidney marker concentration is about 20.2 ng/mL.

In still another aspect, the invention encompasses kits comprising reagents for performing an assay configured to detect WAP four-disulfide core domain protein 2, and a device which contains an encoded calibration curve for correlating results from performing said assay to a concentration of WAP four-disulfide core domain protein 2, wherein the concentration range of said calibration curve comprises a normal concentration of WAP four-disulfide core domain protein 2 and a threshold concentration of WAP four-disulfide core domain protein 2 used to diagnose acute kidney injury.

In another embodiment, the invention encompasses methods of assigning an increased likelihood that a subject having CKD is susceptible to CKD progression, comprising: obtaining a sample of bodily fluid from said subject; performing one or more assays on said sample to determine the presence of one or more biomarkers associated with CKD progression to provide one or more assay results; and assigning an increased likelihood or decreased likelihood of CKD progression to said subject based on the assay result(s).

In certain illustrative embodiments, the one or more biomarkers associated with CKD progression are selected from the group comprising TNFR1a, Troy, NT-proCNP, NGAL 1621-99741, RAGE, Galectin-3, WAP4C, Angiogenic ESAM, and PIGR.

In certain illustrative embodiments, the assigning step comprises comparing each assay result obtained to a corresponding threshold level; and assigning an increased likelihood of CKD progression to a subject when the assay result is greater than the threshold, relative to a risk assigned when the assay result is less than the threshold level, or by assigning a decreased likelihood of CKD progression to a subject when the assay result is less than the threshold, relative to a risk assigned when the assay result is greater than the threshold level.

In certain illustrative embodiments, the threshold level is determined from a first population of subjects diagnosed with CKD and thus susceptible to CKD progression, and the threshold level is selected to separate said population from a second population not diagnosed with CKD.

In certain illustrative embodiments, the threshold level separates said first population from said second population with an odds ratio of at least 2 or more or 0.5 or less.

In certain illustrative embodiments, the threshold level separates said first population from said second population with an odds ratio of at least 3 or more or 0.33 or less.

In certain illustrative embodiments, the body fluid sample is selected from the group consisting of urine, blood, serum, and plasma.

In another embodiment, the invention encompasses methods of assigning an increased likelihood of CKD progression in a subject previously diagnosed with CKD, comprising; performing an assay method that detects one or more biomarkers for CKD progression in a body fluid sample obtained from said subject, thereby providing an assay result; and relating the assay result to the subject's likelihood of CKD progression.

In certain illustrative embodiments, the one or more biomarkers for CKD progression is selected from the group comprising TNFR1a, Troy, NT-proCNP, NGAL 1621-99741, RAGE, Galectin-3, WAP4C, and Angiogenin.

In other illustrative embodiments, the relating step comprises determining a concentration of the one or more biomarkers for CKD progression and relating said concentration to the likelihood of CKD progression in the subject.

In certain illustrative embodiments, the relating step comprises assigning an increased likelihood of CKD progression when the concentration of the one or more biomarkers for CKD progression is greater than a predetermined baseline value for a biomarker for CKD progression.

In other illustrative embodiments, the predetermined baseline value for a biomarker for CKD progression is determined by performing an assay method that detects the one or more biomarkers for CKD progression of claim 2 in a body fluid sample obtained from said subject at a time earlier than the time at which the body fluid sample used to provide the assay result was obtained.

In certain illustrative embodiments, the predetermined baseline value for a biomarker for CKD progression is determined from a first population of subjects diagnosed with CKD and a second population of subjects not diagnosed with CKD.

In certain illustrative embodiments, the body fluid sample is selected from the group consisting of urine, blood, serum, and plasma.

In certain illustrative embodiments, an increase in the level of a biomarker for CKD progression of about 2 fold over the baseline value is indicative of an increased likelihood of CKD progression.

In certain illustrative embodiments, an increase in the level of a biomarker for CKD progression of about 4 fold over the baseline value Is indicative of an increased likelihood of CKD progression.

In other illustrative embodiments, an increase in the level of a biomarker for CKD progression of in a sample obtained at a later time compared to the level of a biomarker for CKD progression obtained from said subject at an earlier time is indicative of an increased likelihood of CKD progression.

In another embodiment, the invention encompasses kits comprising: reagents for performing an assay configured to detect one or more biomarkers for CKD progression; and a device which contains an encoded calibration curve for correlating results from performing said assay to a concentration of one or more markers, wherein the concentration range of said calibration curve comprises a normal concentration of biomarker for CKD progression and a threshold concentration of biomarker for CKD progression used to assign an increased likelihood of CKD progression.

### BRIEF DESCRIPTION OF THE FIGURES

FIG. 1 illustrates an ROC (Receiver Operating Characteristic) curve of Basel V AKI (consecutive), D @t=0: specificity and sensitivity vs. admission [WAP4C], wherein the horizontal axis of the ROC curve represents 1-specificity (which increases with the rate of false positives), and the vertical axis of the curve represents sensitivity (which increases with the rate of true positives), and the area under the ROC curve is a measure of the probability that the measured marker level will allow correct identification of a disease or condition and accordingly can be used to determine the effectiveness of the test.

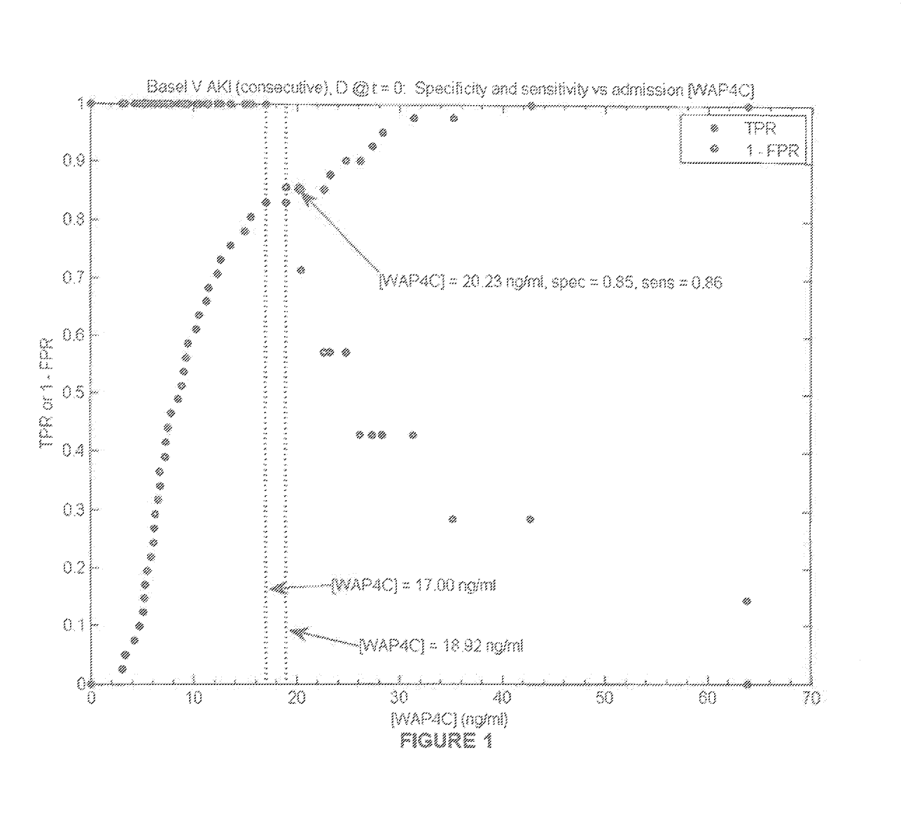

### DETAILED DESCRIPTION OF THE INVENTION

The present invention encompasses methods and compositions for diagnosis, prognosis, and determination of treatment regimens in subjects suffering from renal failure.

As described herein, the invention relates in part to assigning an outcome risk (e.g., worsening renal function, risk of re-hospitalization, and/or a mortality risk) to a subject based, at least in part, on the results obtained from an assay that detects WAP four-disulfide core domain protein 2 performed on a body fluid sample obtained from a subject.

If the sample tested is obtained from the subject at a time t, the phrase “short term risk” refers to a 7-day (168 hour) period measured from time t. Thus, the risk is a likelihood that the subject will suffer from deterioration of one or more of measures of renal function, will require re-hospitalization, or will die, in a window beginning at time t and ending 168 hours later. Suitable measures of cardiac function include one or more of: dyspnea (at rest or exertional), orthopnea, pulmonary edema, SaO₂ level, dizziness or syncope, chest pain, systolic blood pressure, hypoperfusion, edema, compensation status (that is, a change from compensated to decompensated, or vice versa), end-diastolic function, end-systolic function, ventricular filling, flow across the mitral valve, left ventricular ejection fraction (LVEF), results of stress testing, results of an imaging study such as a cardiac CT, ultrasound, or MRI, NYHA or American College of Cardiology heart failure classification, etc. These characteristics, and methods for their assessment, are well known in the art. See, e.g., Harrison's Principles of Internal Medicine, 16th ed. McGraw-Hill, 2005, pages 1361-1377, which is hereby incorporated by reference in its entirety. This list is not meant to be limiting.

More preferably, the risk is a likelihood that the subject will suffer from deterioration of one or more of these measures of renal function, will require rehospitalisation, or will die, in a 96 hour window beginning at time t, and most preferably the risk is a likelihood that the subject will suffer from deterioration of one or more of these measures of renal function, or a likelihood that the subject will die, in a window of between 48 and 84 hours beginning at time t. The term “deterioration” as used herein refers to a worsening change in a parameter at a later time, relative to a measure of the same parameter earlier in the same subject, and is the opposite of “improvement.” For example, “deterioration in renal function” as used herein refers to a later change in the subject from an asymptomatic state.

The terms “marker” and “biomarker” as used herein refers to proteins, polypeptides, glycoproteins, proteoglycans, lipids, lipoproteins, glycolipids, phospholipids, nucleic acids, carbohydrates, etc. or small molecules to be used as targets for screening test samples obtained from subjects. “Proteins or polypeptides” used as markers in the present invention are contemplated to include any fragments thereof, in particular, immunologically detectable fragments. Markers can also include clinical “scores” such as a pre-test probability assignment, a pulmonary hypertension “Daniel” score, an NIH stroke score, a Sepsis Score of Elebute and Stoner, a Duke Criteria for Infective Endocarditis, a Mannheim Peritonitis Index, an “Apache” score, etc.

As used herein, the terms “WAP four-disulfide core domain protein 2” “WAP4C” and “HE4” refer to one or more polypeptides, isoforms, splice variants or fragments thereof present in a biological sample that are derived from a WAP four-disulfide core domain protein 2 precursor. The human precursor (Swiss-Prot entry Q14508) has the following sequence (SEQ ID NO: 1):

```
                10 20 30 40
MPACRLGPLA AALLLSLLLF GFTLVSGTGA EKTGVCPELQ
                50 60 70 80
ADQNCTQECV SDSECADNLK CGSAGCATFC SLPNDKEGSC
                90 100 110 120
PQVNINFPQL GLCRDQCQVD SQCPGQMKCC RNGCGKVSCV
```

The following domains have been Identified in WAP four-disulfide core domain protein 2:

| Residues | Length | Domain ID |
|---|---|---|
| 1-30 | 30 | signal sequence |
| 31-124 | 94 | WAP four-disulfide core domain protein 2 |

And the following alternative forms derived from the WAP four-disulfide core domain protein 2 precursor have been described:

| | | |
|---|---|---|
| 2-23 | 22 | → LQVQNLPVSPLPTYPYSFF YP |
| | | (SEQ ID NO: 2) in isoform 2. |
| 24-74 | 51 | Missing in isoform 2. |
| 27-74 | 48 | Missing in isoform 3. |
| 71-79 | 9 | → LLCPNGQLAE (SEQ ID NO: 3) |
| | | in isoform 4. |
| 75-102 | 28 | → ALFHWHLKTRRLWEISGPRP RRPTWDSS |
| | | (SEQ ID NO: 4) in isoform 5. |
| 80-124 | 45 | Missing in isoform 4. |
| 103-124 | 22 | Missing in isoform 5. |

Because production of marker fragments is an ongoing process that may be a function of, inter alia, the elapsed time between onset of an event triggering marker release into the tissues and the time the sample is obtained or analyzed; the elapsed time between sample acquisition and the time the sample is analyzed; the type of tissue sample at issue; the storage conditions; the quantity of proteolytic enzymes present; etc., it may be necessary to consider this degradation when both designing an assay for one or more markers, and when performing such an assay, in order to provide an accurate prognostic or diagnostic result. In addition, individual antibodies that distinguish amongst a plurality of marker fragments may be individually employed to separately detect the presence or amount of different fragments. The results of this individual detection may provide a more accurate prognostic or diagnostic result than detecting the plurality of fragments in a single assay.

As used herein, the terra “relating a signal to the presence or amount” of an analyte reflects this understanding. Assay signals are typically related to the presence or amount of an analyte through the use of a standard curve calculated using known concentrations of the analyte of interest. As the term is used herein, an assay is “configured to detect” an analyte if an assay can generate a detectable signal indicative of the presence or amount of a physiologically relevant concentration of the analyte.

Because an antibody epitope is on the order of 8 amino acids, an immunoassay configured to detect a marker of interest will also detect polypeptides related to the marker sequence, so long as those polypeptides contain the epitope(s) necessary to which the antibody or antibodies used in the assay will bind.

The term “related marker” as used herein with regard to a biomarker such as one of the markers described herein refers to one or more fragments, variants, etc., of a particular marker or its biosynthetic parent that may be detected as a surrogate for the marker itself or as independent biomarkers. The term also refers to one or more polypeptides present in a biological sample that are derived from the biomarker precursor complexed to additional species, such as binding proteins, receptors, heparin, lipids, sugars, etc.

In this regard, the skilled artisan will understand that the signals obtained from an immunoassay are a direct result of complexes formed between one or more antibodies and the target bimolecule (i.e., the analyte) and polypeptides containing the necessary epitope(s) to which the antibodies bind. While such assays may detect the full length biomarker and the assay result be expressed as a concentration of a biomarker of interest, the signal from the assay is actually a result of all such “immunoreactive” polypeptides present in the sample. Expression of biomarkers may also be determined by means other than immunoassays, Including protein measurements (such as dot blots, western blots, chromatographic methods, mass spectrometry, etc.) and nucleic acid measurements mRNA quatitation). This list is not meant to be limiting.

Preferred assays are “configured to detect” a particular marker. That an assay is “configured to detect” a marker means that an assay can generate a detectable signal Indicative of the presence or amount of a physiologically relevant concentration of a particular marker of interest. Such an assay may, but need not, specifically detect a particular marker (i.e., detect a marker but not some or all related markers). Because an antibody epitope is on the order of 8 amino acids, an immunoassay will detect other polypeptides (e.g., related markers) so long as the other polypeptides contain the epitope(s) presented in such a way as is necessary for the antibody (antibodies) used in the assay to bind. Such other polypeptides are referred to as being “immunologically detectable” In the assay, and would include various isoforms (e.g., splice variants). In the case of a sandwich immunoassay, related markers must contain at least the two distinct and accessible epitopes to which at least two distinct antibodies can bind in order for the marker to be detected. Preferred immunologically detectable fragments comprise at least 8 contiguous residues of the marker or its biosynthetic parent.

The term “test sample” as used herein refers to a sample of bodily fluid obtained for the purpose of diagnosis, prognosis, or evaluation of a subject of interest, such as a patient. In certain embodiments, such a sample may be obtained for the purpose of determining the outcome of an ongoing condition or the effect of a treatment regimen on a condition. Preferred test samples include blood, serum, plasma, cerebrospinal fluid, urine, saliva, sputum, and pleural effusions. In addition, one of skill in the art would realize that some test samples would be more readily analysed following a fractionation or purification procedure, for example, separation of whole blood into serum or plasma components.

As used herein, a “plurality” refers to at least two. Preferably, a plurality refers to at least 3, more preferably at least 5, even more preferably at least 10, even more preferably at least 15, and most preferably at least 20. In particularly preferred embodiments, a plurality is a large number, i.e., at least 100. The term “subject” as used herein refers to a human or non-human organism.

Thus, the methods and compositions described herein are applicable to both human and veterinary disease. Further, while a subject is preferably a living organism, the invention described herein may be used in post-mortem analysis as well. Preferred subjects are “patients,” (i.e., living humans that are receiving medical care for a disease or condition). This includes persons with no defined illness who are being investigated for signs of pathology.

The term “diagnosis” as used herein refers to methods by which the skilled artisan can estimate and/or determine whether or not a patient is suffering from a given disease or condition. The skilled artisan often makes a diagnosis on the basis of one or more diagnostic indicators (i.e., a marker), the presence, absence, amount, or change in amount of which is indicative of the presence, severity, or absence of the condition. The term “diagnosis” does not refer to the ability to determine the presence or absence of a particular disease with 100% accuracy, or even that a given course or outcome is more likely to occur than not Instead, the skilled artisan will understand that the term “diagnosis” refers to an increased probability that a certain disease is present in the subject.

Similarly, a prognosis is often determined by examining one or more “prognostic indicators.” These are markers, the presence or amount of which in a patient (or a sample obtained from the patient) signal a probability that a given course or outcome will occur.

For example, when one or more prognostic indicators reach a sufficiently high level in samples obtained from such patients, the level may signal that the patient is at an increased probability for experiencing morbidity or mortality in comparison to a similar patient exhibiting a lower marker level. A level or a change in level of a prognostic indicator, which in turn is associated with an increased probability of morbidity or death, is referred to as being “associated with an increased predisposition to m adverse outcome” in a patient.

The term “correlating” or “relating” as used herein in reference to the use of markers, refers to comparing the presence or amount of the marker(s) in a patient to its presence or amount in persons known to suffer from, or known to be at risk of, a given condition; or in persons known to be free of a given condition. As discussed above, a marker level in a patient sample can be compared to a level known to be associated with a specific diagnosis. The sample's marker level is said to have been correlated with a diagnosis; that is, the skilled artisan can use the marker level to determine whether the patient suffers from a specific type diagnosis, and respond accordingly. Alternatively, the sample's marker level can be compared to a marker level known to be associated with a good outcome (e.g., the absence of disease, etc.). In preferred embodiments, a profile of marker levels are correlated to a global probability or a particular outcome using ROC curves.

In certain embodiments, the methods described herein comprise the comparison of an assay result to a corresponding baseline result. The term “baseline result” as used herein refers to an assay value that is used as a comparison value (that is, to which a test result is compared). In practical terms, this means that a marker is measured in a sample from a subject and the result is compared to the baseline result. A value above the baseline Indicates a first likelihood of a diagnosis or prognosis, and a value below the baseline indicates a second likelihood of a diagnosis or prognosis.

A baseline can be selected in a number of manners well known to those of skill in the art. For example, data for a marker or markers (e.g., concentration in a body fluid, such as urine, blood, serum, or plasma) may be obtained from a population of subjects.

The population of subjects is divided into at least two subpopulations. The first subpopulation includes those subjects who have been confirmed as having a disease, outcome, or, more generally, being in a first condition state. For example, this first subpopulation of patients may be those diagnosed with renal failure, and that suffered from a worsening of renal function. For convenience, subjects in this first subpopulation will be referred to as “diseased,” although in fact, this subpopulation is actually selected for the presence of a particular characteristic of Interest. The second subpopulation of subjects is formed from the subjects that do not fall within the first sub-population.

Subjects in this second set will hereinafter be referred to as “non-diseased.” A baseline result may then be selected to distinguish between the diseased and non-diseased subpopulation with an acceptable specificity and sensitivity. Changing the baseline merely trades off between the number of false positives and the number of false negatives resulting from the use of the particular marker under study. The effectiveness of a test having such an overlap is often expressed using a ROC (Receiver Operating Characteristic) curve. ROC curves are well known to those skilled in the art. The horizontal axis of the ROC curve represents (1-specificity), which increases with the rate of false positives. The vertical axis of the curve represents sensitivity, which increases with the rate of true positives. Thus, for a particular cutoff selected, the value of (1-specificity) may be determined, and a corresponding sensitivity may be obtained. The area under the ROC curve is a measure of the probability that the measured marker level will allow correct identification of a disease or condition. Thus, the area under the ROC curve can be used to determine the effectiveness of the test.

In an alternative, an individual subject may provide their own baseline, in that a temporal change is used to Indicate a particular diagnosis or prognosis. For example, one or more markers may be determined at an initial time to provide one or more baseline results, and then again at a later time, and the change (or lack thereof) in the marker level(s) over time determined, in such embodiments, an increase in the marker from the initial time to the second time may be indicative of a particular prognosis, or a particular diagnosis, etc. Likewise, a decrease in the marker from the initial time to the second time may be indicative of a particular prognosis, or a particular diagnosis, etc. In such an embodiment, a plurality of markers need not change in concert with one another. Temporal changes in one or more markers may also be used together with single time point marker levels compared to a population-based baseline.

In certain embodiments, a baseline marker level is established for a subject, and a subsequent assay result for the same marker is determined. That subsequent result is compared to the baseline result, and a value above the baseline indicates worsening cardiac function, worsening renal function, or both, relative to a value below the baseline. Similarly, a value below the baseline indicates improved cardiac function, improved renal function, or both, relative to a value above the baseline.

In certain embodiments, a baseline marker level is established for a subject, and a subsequent assay result for the same marker is determined. That subsequent result is compared to the baseline result, and a value above the baseline indicates an increased mortality risk, relative to a value below the baseline. Similarly, a value below the baseline indicates a decreased mortality risk, relative to a value above the baseline.

As discussed herein, the measurement of the level of a single marker may be augmented by additional markers. For example, other markers related to blood pressure regulation, including other natriuretic peptides and/or their related markers may be used together with, or separately from, BNP and/or Its related markers. Suitable assays include, bur are not limited to, assays that detect ANP, proANP, NT-proANP, CNP, Kininogen, CGRP II, urotensin II, BNP, NT-proBNP, proBNP, calcitonin gene related peptides arg-Vasopressin, Endothelin-1 (and/or Big ET-1), Endothelin-2 (and/or Big ET-2), Endothelin-3 (and/or Big ET-3), procalcitonin, calcyphosine, adrenomeduilin, aldosterone, angiotensin 1 (and/or angiotensinogen 1), angiotensin 2 (and/or angiotensinogen 2), angiotensin 3 (and/or angiotensinogen 3), Bradykinin, Tachykinin-3, calcitonin. Renin, Urodilatin, and Ghrelin, and/or one or more markers related thereto.

Various clinical variables may also be utilized as variables in the methods described herein. Examples of such variables include urine output levels, age, the presence or absence of one or more cardiovascular or renal risk factors such as diabetes, hypertension, smoking status, etc. This list is not meant to be limiting.

Suitable methods for combining markers into a single composite value that may be used as if it is a single marker are described in detail in U.S. Provisional Patent Application No. 60/436,392 filed Dec. 24, 2002, PCT application US03/41426 filed Dec. 23, 2003, U.S. patent application Ser. No. 10/331,127 filed Dec. 27, 2002, and PCT application No, US03/41453, each of which is hereby incorporated by reference in its entirety, including all tables, figures, and claims. In an alternative, assay results may be used in an “n-of-m” type of approach. Using a two marker example of such methods, when either marker above its corresponding baseline value may signal a renal failure diagnosis or an increased risk of an adverse outcome (in n-of-m terms, this is a “1-of-2” result). If both are above the corresponding baselines (a “2-of-2” result), an even greater confidence in the subject's status may be indicated.

The sensitivity and specificity of a diagnostic and/or prognostic test depends on more than just the analytical “quality” of the test-they also depend on the definition of what constitutes an abnormal result. In practice, Receiver Operating Characteristic curves, or “ROC” curves, are typically calculated by plotting the value of a variable versus its relative frequency in “normal” and “disease” populations. For any particular marker, a distribution of marker levels for subjects with and without a “disease” will likely overlap. Under such conditions, a test does not absolutely distinguish normal from disease with 100% accuracy, and the area of overlap indicates where the test cannot distinguish normal from disease. A threshold is selected, above which (or below which, depending on how a marker changes with the disease) the test is considered to be abnormal and below which the test is considered to be normal. The area under the ROC curve is a measure of the probability that the perceived measurement will allow correct identification of a condition. ROC curves can be used even when test results don't necessarily give an accurate number. As long as one can rank results, one can create a ROC curve. For example, results of a test on “disease” samples might be ranked according to degree (say 1=low, 2=normal, and 3=high). This ranking can be correlated to results In the “normal” population, and a ROC curve created, These methods are well known in the art. See, e.g., Hanley et al., Radiology 143: 29-36 (1982).

Measures of test accuracy may also be obtained as described in Fischer et al., Intensive Care Med. 29: 1043-51,2003, and used to determine the effectiveness of a given marker or panel of markers. These measures include sensitivity and specificity, predictive values, likelihood ratios, diagnostic odds ratios, and ROC curve areas. As discussed above, preferred tests and assays exhibit one or more of the following results on these various measures.

Preferably, a baseline is chosen to exhibit at least about 70% sensitivity, more preferably at least about 80% sensitivity, even more preferably at least about 85% sensitivity, still more preferably at least about 90% sensitivity, and most preferably at least about 95% sensitivity, combined with at least about 70% specificity, more preferably at least about 80% specificity, even more preferably at least about 85% specificity, still more preferably at least about 90% specificity, and most preferably at least about 95% specificity. In particularly preferred embodiments, both the sensitivity and specificity are at least about 75%, more preferably at least about 80%, even more preferably at least about 85%, still more preferably at least about 90%, and roost preferably at least about 95%, The term “about” in this context refers to +/−5% of a given measurement.

In other embodiments, a positive likelihood ratio, negative likelihood ratio, odds ratio, or hazard ratio is used as a measure of a test's ability to predict risk or diagnose a disease. In the case of a positive likelihood ratio, a value of 1 indicates that a positive result is equally likely among subjects in both the “diseased” and “control” groups; a value greater than 1 indicates that a positive result is more likely in the diseased group; and a value less than 1 indicates that a positive result is more likely in the control group.

In the case of a negative likelihood ratio, a value of 1 indicates that a negative result is equally likely among subjects in both the “diseased” and “control” groups; a value greater than 1 indicates that a negative result is more likely in the test group; and a value less than 1 indicates that a negative result is more likely in the control group, in certain preferred embodiments, markers and/or marker panels are preferably selected to exhibit a positive or negative likelihood ratio of at least about 1.5 or more or about 0.67 or less, more preferably at least about 2 or more or about 0.5 or less, still more preferably at least about 5 or more or about 0.2 or less, even more preferably at least about 10 or more or about 0.1 or less, and most preferably at least about 20 or more or about 0.05 or less. The term “about” in this context refers to +/−5% of a given measurement.

In the case of an odds ratio, a value of 1 indicates that a positive result is equally likely among subjects in both the “diseased” and “control” groups; a value greater than 1 indicates that a positive result is more likely in the diseased group; and a value less than 1 indicates that a positive result is more likely in the control group. In certain preferred embodiments, markers and/or marker panels are preferably selected to exhibit an odds ratio of at least about 2 or more or about 0.5 or less, more preferably at least about 3 or more or about 0.33 or less, still more preferably at least about 4 or more or about 0.25 or less, even more preferably at least about 5 or more or about 0.2 or less, and most preferably at least about 10 or more or about 0.1 or less. The term “about” in this context refers to +/−5% of a given measurement.

In the case of a hazard ratio, a value of 1 indicates that the relative risk of an endpoint (e.g., death) is equal in both the “diseased” and “control” groups; a value greater than 1 indicates that the risk is greater in the diseased group; and a value less than 1 indicates that the risk is greater in the control group. In certain preferred embodiments, markers and/or marker panels are preferably selected to exhibit a hazard ratio of at least about 1.1 or more or about 0.91 or less, more preferably at least about 1.25 or more or about 0.8 or less, still more preferably at least about 1.5 or more or about 0.67 or less, even more preferably at least about 2 or more or about 0.5 or less, and most preferably at least about 2.5 or more or about 0.4 or less. The term “about” in this context refers to +/−5% of a given measurement.

Numerous methods and devices are well known to the skilled artisan for the detection and analysis of the markers of the instant invention. With regard to polypeptides or proteins in patient test samples, immunoassay devices and methods are often used. See, e.g., U.S. Pat. Nos. 6,143,576; 6,113,855; 6,019,944; 5,985,579; 5,947,124; 5,939,272; 5,922,615; 5,885,527; 5,851,776; 5,824,799; 5,679,526; 5,525,524; and 5,480,792, each of which is hereby incorporated by reference in its entirety, including all tables, figures and claims. These devices and methods can utilize labeled molecules in various sandwich, competitive, or non-competitive assay formats, to generate a signal that Is related to the presence or amount of an analyte of interest.

Additionally, certain methods and devices, such as biosensors and optical immunoassays, may be employed to determine the presence or amount of analytes without the need for a labeled molecule. See, e.g., U.S. Pat. Nos. 5,631,171; and 5,955,377, each of which is hereby incorporated by reference in its entirety. Including all tables, figures and claims.

One skilled in the art also recognizes that robotic instrumentation Including but not limited to Beckman Access, Abbott AxSym, Roche ElecSys, Dade Behring Stratus systems are among the immunoassay analyzers that are capable of performing the immunoassays taught herein.

Preferably the markers are analyzed using an immunoassay, and most preferably sandwich Immunoassay, although other methods are well known to those skilled in the art (for example, the measurement of marker RNA levels). The presence or amount of a marker is generally determined using antibodies specific for each marker and detecting specific binding. Any suitable immunoassay may be utilized, for example, enzyme-linked immunoassays (ELISA), radioimmunoassays (RIAs), competitive binding assays, and the like. Specific immunological binding of the antibody to the marker can be detected directly or indirectly. Biological assays such as immunoassays require methods for detection, and one of the most common methods for quantitation of results is to conjugate an enzyme, fluorophore or other molecule to form an antibody-label conjugate,

Detectable labels may include molecules that are themselves detectable (e.g., fluorescent moieties, electrochemical labels, metal chelates, etc.) as well as molecules that may be indirectly detected by production of a detectable reaction product (e.g., enzymes such as horseradish peroxidase, alkaline phosphatase, etc.) or by a specific binding molecule which itself may be detectable (e.g., biotin, digoxigenin, maltose, oligohistidine, 2,4-dintrobenzene, phenylarsenate, ssDNA, dsDNA, etc.). Particularly preferred detectable labels ate fluorescent latex particles such as those described in U.S. Pat. Nos. 5,763,189, 6,238,931, and 6,251,687; and international Publication WO95/08772, each of which is hereby incorporated by reference in its entirety. Exemplary conjugation to such particles is described hereinafter. Direct labels include fluorescent or luminescent tags, metals, dyes, radionuclides, and the like, attached to the antibody. Indirect labels include various enzymes well known In the art, such as alkaline phosphatase, horseradish peroxidase and the like.

The use of immobilized antibodies specific for the markers is also contemplated by the present invention. The term “solid phase” as used herein refers to a wide variety of materials including solids, semi-solids, gels, films, membranes, meshes, felts, composites, particles, papers and the like typically used by those of skill in the art to sequester molecules. The solid phase can be non-porous or porous. Suitable solid phases include those developed and/or used as solid phases in solid phase binding assays. See, e.g., chapter 9 of Immunoassay, E. P. Dianiandis and T. K. Christopoulos eds., Academic Press: New York, 1996, hereby incorporated by reference. Examples of suitable solid phases include membrane filters, cellulose-based papers, beads (including polymeric, latex and paramagnetic particles), glass, silicon wafers, microparticles, nanoparticies, TentaGels, AgroGels, PEG A gels, SPOCC gels, and multiple-well plates. See, e.g., Leon et al., Bioorg. Med, Chem. Lett. 8: 2997, 1998; Kessler et at, Agnew. Chem. Int. Ed. 40: 165, 2001; Smith et al., J. Comb, Med. 1: 326, 1999; Orain et al., Tetrahedron Lett, 42: 515, 2001; Papanikos et al., J. Am, Chem, Soc. 123: 2176, 2001; Gottschling et al., Bioorg. Med. Chem. Lett 11: 2997, 2001. The antibodies could be immobilized onto a variety of solid supports, such as magnetic or chromatographic matrix particles, the surface of an assay place (such as microliter wells), pieces of a solid substrate material or membrane (such as plastic, nylon, paper), and the like. An assay strip could be prepared by coating the antibody or a plurality of antibodies in an array on solid support. This strip could then be dipped into the test sample and then processed quickly through washes and detection steps to generate a measurable signal, such as a colored spot. When multiple assays are being performed, a plurality of separately addressable locations, each corresponding to a different marker and comprising antibodies that bind the appropriate marker, can be provided on a single solid support. The term “discrete” as used herein refers to areas of a surface that are non-contiguous. That is, two areas are discrete from one another if a border that is not part of either area completely surrounds each of the two areas. The term “independently addressable” as used herein refers to discrete areas of a surface from which a specific signal may be obtained.

For separate or sequential assay of markers, suitable apparatuses include clinical laboratory analyzers such as the ElecSys (Roche), the AxSym (Abbott), the Access Beckman), the ADVIAÒ CENTAURÒ (Bayer) immunoassay systems, the NICHOLS ADVANTAGEÒ (Nichols Institute) immunoassay system, etc. Preferred apparatuses perform simultaneous assays of a plurality of markers using a single test device.

Particularly useful physical formats comprise surfaces having a plurality of discrete, addressable locations for the detection of a plurality of different analytes. Such formats include protein microarrays, or “protein chips” (see, e.g., Ng and Ilag, J. Cell Mol. Med. 6: 329-340 (2002)) and certain capillary devices (see, e.g., U.S. Pat. No. 6,019,944). In these embodiments, each discrete surface location may comprise antibodies to immobilize one or more arsalyte(s) (e.g., a marker) for detection at each location.

Surfaces may alternatively comprise one or more discrete panicles (e.g., microparticles or nanoparticles) immobilized at discrete locations of a surface, where the microparticles comprise antibodies to immobilize one analyte (e.g., a marker) for detection.

Preferred assay devices of the present invention will comprise, for one or more assays, a first antibody conjugated to a solid phase and a second antibody conjugated to a signal development element. Such assay devices are configured to perform a sandwich immunoassay for one or more analytes. These assay devices will preferably further comprise a sample application zone, and a flow path from the sample application zone to a second device region comprising the first antibody conjugated to a solid phase.

Flow of a sample in an assay device along the flow path may be driven passively (e.g., by capillary, hydrostatic, or other forces that do not require further manipulation of the device once sample is applied), actively (e.g., by application offeree generated via mechanical pumps, electroosmotic pumps, centrifugal force, increased air pressure, etc.), or by a combination of active and passive driving forces. Most preferably, sample applied to the sample application zone will contact both a first antibody conjugated to a solid phase and a second antibody conjugated to a signal development element along the flow path (sandwich assay format). Additional elements, such as filters to separate plasma or serum from blood, mixing chambers, etc., may be included as required by the artisan.

Exemplary devices are described in Chapter 41, entitled “Near Patient Tests: Triage® Cardiac System,” in The Immunoassay Handbook, 2nd ed., David Wild, ed., Nature Publishing Group, 2001, which is hereby incorporated by reference in its entirety.

The analysis of markers could be carried out in a variety of physical formats as well For example, the use of microliter plates or automation could be used to facilitate the processing of large numbers of test samples. Alternatively, single sample formats could be developed to facilitate immediate treatment and diagnosis in a timely fashion, for example, in ambulatory transport or emergency room settings. In another embodiment, the present invention provides a kit for the analysis of markers. Such a kit preferably comprises devises and reagents for the analysis of at least one test sample and instructions for performing the assay(s) of interest. Optionally the kits may contain one or more means for using information obtained from immunoassays or other specific binding assays performed for a marker panel to rule in or out certain diagnoses or prognoses. Other measurement strategies applicable to the methods described herein include chromatography (e.g., HPLC), mass spectrometry, receptor based assays, and combinations of the foregoing.

The term “antibody” as used herein refers to a peptide or polypeptide derived from, modeled after or substantially encoded by an Immunoglobulin gene or immunoglobulin genes, or fragments thereof, capable of specifically binding an antigen or epitope. See, e.g. Fundamental Immunology, 3rd Edition, W. E. Paul, ed., Raven Press, N.Y. (1993); Wilson (1994) J. Immunol. Methods 175:267-273; Yarmush (1992) J. Biochem, Biophys. Methods 25:85-97. The term antibody includes antigen-binding portions, i.e., “antigen binding sites” (e.g., fragments, subsequences, complementarity determining regions (CDRs)) that retain capacity to bind antigen, including (i) a Fab fragment, a monovalent fragment consisting of the VL, VH, CL and CHI domains; (ii) a F(ab′)2 fragment, a bivalent fragment comprising two Fab fragments linked by a disulfide bridge at the hinge region; (iii) a Fd fragment consisting of the VH and CHI domains; (iv) a Fv fragment consisting of the VL and VR domains of a single arm of an antibody, (v) a dAb fragment (Ward et al., (1939) Nature 341:544-546), which consists of a VH domain; and (vi) an isolated complementarity determining region (CDR). Single chain antibodies are also included by reference in the term “antibody.” While the present invention is described in detail in terms of immunologic detection of an analyte, other marker binding partners such as aptamers, receptors, binding proteins, etc., may be used in a similar fashion to antibodies in providing an assay.

Preferably, an antibody or other binding partner used in an assay is selected that specifically binds a marker of interest. The term “specifically binds” is not Intended to indicate that an antibody/binding partner binds exclusively to its intended target. Rather, an antibody/binding partner “specifically binds” if its affinity for its intended target is about 5-fold greater when compared to its affinity for a non-target molecule. Preferably the affinity of the antibody will be at least about 5 fold, preferably 10 fold, more preferably 25-fold, even more preferably 50-fold, and most preferably 100-fold or more, greater for a target molecule than its affinity for a non-target molecule. The affinity of a targeting agent for its target molecule is preferably at least about 1×10⁻⁶ moles/liter, is more preferably at least about 1×10⁻⁷ moles/liter, is even more preferably at least about 1×10⁻⁸ moles/liter, Is yet even more preferably at least about 1×10⁻⁹ moles/liter, and Is most preferably at least about 1×10⁻¹⁰ moles/liter. In preferred embodiments, specific binding between an antibody or other binding agent and an antigen means a binding affinity of at least 10⁶ M⁻¹. Preferred antibodies bind with affinities of at least about 10⁷ M⁻¹, and preferably between about 10⁸ M⁻¹ to about 10⁹ M⁻¹ about 10⁹ M⁻¹ to about 10¹⁰ M⁻¹, or about 10 ¹⁰ M⁻¹ to about 10¹¹ M⁻¹.

Affinity is calculated as K_{d}=K_{off}/kₒₙ (k_{off} is the dissociation rate constant, kₒₙ is the association rate constant and K_{d} is the equilibrium constant. Affinity can be determined at equilibrium by measuring the fraction bound (r) of labeled ligand at various concentrations (c). The data are graphed using the Scatchard equation: r/c=K(nr):

where

r=moles of bound ligand/mole of receptor at equilibrium;

c=free ligand concentration at equilibrium;

K=equilibrium association constant; and

n=number of ligand binding sites per receptor molecule

By graphical analysis, r/c is plotted on the Y-axis versus r on the X-axis thus producing a Scatchard plot. The affinity is the negative slope of the line. k_{off} can be determined by competing bound labeled ligand with unlabeled excess ligand (see, e.g., U.S. Pat No. 6,316,409).

Antibody affinity measurement by Scatchard analysis is well known in the art. See, e.g., van Erp et al., J, Immunoassay 12: 425-43,1991; Nelson and Griswold, Comput. Methods Programs Biomed. 27: 65-8, 1988. The generation and selection of antibodies may be accomplished several ways. For example, one way is to purify polypeptides of Interest or to synthesize the polypeptides of interest using, e.g., solid phase peptide synthesis methods well known in the art. See, e.g., Guide to Protein Purification, Murray P. Deutcher, ed., Meth. Enzymol. Vol 182 (1990); Solid Phase Peptide Synthesis, Greg B. Fields ed., Meth. Enzymol. Vol 289 (1997); Kiso et al., Chem. Pharm. Bull. (Tokyo) 38: 1192-99, 1990; Mostafavi et al., Biomed. Pept. Proteins Nucleic Acids 1: 255-60,1995; Fujiwara et al. Chem, Pharm. Bull. (Tokyo) 44: 1326-31, 1996. The selected polypeptides may then be injected, for example. Into mice or rabbits, to generate polyclonal or monoclonal antibodies. One skilled in the art will recognize that many procedures are available for the production of antibodies, for example, as described in Antibodies, A Laboratory Manual, Ed Harlow and David Lane, Cold Spring Harbor Laboratory (1988), Cold Spring Harbor, N.Y. One skilled in the art will also appreciate that binding fragments or Fab fragments which mimic antibodies can also be prepared from genetic information by various procedures (Antibody Engineering: A Practical Approach (Borrebaeck, C., ed.), 1995, Oxford University Press, Oxford; J. Immunol. 149, 3914-3920 (1992)).

In addition, numerous publications have reported the use of phage display technology to produce and screen libraries of polypeptides for binding to a selected target. See, e.g., Cwirla et al., Froc. Natl. Acad. Sci. USA 87, 6378-82, 1990; Devlin et al., Science 249,404-6, 1990, Scott and Smith, Science 249, 386-88, 1990; and Ladner et al., U.S. Pat. No. 5,571,698. A basic concept of phage display methods is the establishment of a physical association between DNA encoding a polypeptide to be screened and the polypeptide. This physical association is provided by the phage particle, which displays a polypeptide as part of a capsid enclosing the phage genome which encodes the polypeptide. The establishment of a physical association between polypeptides and their genetic material allows simultaneous mass screening of very large numbers of phage hearing different polypeptides. Phage displaying a polypeptide with affinity to a target bind to the target and these phage are enriched by affinity screening to the target. The identity of polypeptides displayed from these phage can be determined from their respective genomes. Using these methods a polypeptide identified as having a binding affinity for a desired target can then be synthesized in bulk by conventional means. See, e.g., U.S. Pat. No. 6,057,098, which is hereby incorporated in its entirety, including all tables, figures, and claims.

The antibodies that are generated by these methods may then be selected by first screening tor affinity and specificity with the purified polypeptide of interest and, if required, comparing the results to the affinity and specificity of the antibodies with polypeptides that are desired to be excluded from binding. The screening procedure can involve immobilization of the purified polypeptides in separate wells of microliter plates.

The solution containing a potential antibody or groups of antibodies is then placed into the respective microliter wells and Incubated for about 30 min to 2 h. The microliter wells are then washed and a labeled secondary antibody (for example, an anti-mouse antibody conjugated to alkaline phosphatase if the raised antibodies are mouse antibodies) is added to the wells and incubated for about 30 min and then washed. Substrate is added to the wells and a color reaction will appear where antibody to the immobilized polypeptide(s) is present.

The antibodies so identified may then be further analyzed tor affinity and specificity in the assay design selected. In the development of immunoassays for a target protein, the purified target protein acts as a standard with which to judge the sensitivity and specificity of the immunoassay using the antibodies that have been selected. Because the binding affinity of various antibodies may differ; certain antibody pairs (e.g., in sandwich assays) may interfere with one another sterically, etc., assay performance of an antibody may be a more important measure than absolute affinity and specificity of an antibody,

Those skilled in the art will recognize that many approaches can be taken in producing antibodies or binding fragments and screening and selecting for affinity and specificity for the various polypeptides, but these approaches do not change the scope of the invention.

Nucleic acid aptamers are nucleic acid species that have been engineered through repeated rounds of in vitro selection or equivalently, SELEX (systematic evolution of ligands by exponential enrichment) to bind to various molecular targets such as small molecules, proteins, nucleic acids, and even ceils, tissues and organisms. Peptide aptamers are proteins that are designed to interfere with other protein interactions Inside cells. They consist of a variable peptide loop attached at both ends to a protein scaffold.

This double structural constraint greatly increases the binding affinity of the peptide aptamer to levels comparable to an antibody's (nanomolar range). Aptamers are useful in biotechnological and therapeutic applications as they offer molecular recognition properties that rival that of the commonly used biomolecule, antibodies. In addition to their discriminate recognition, aptamers offer advantages over antibodies as they can be engineered completely in a test tube, are readily produced by chemical synthesis, possess desirable storage properties, and elicit little or no immunogenicity in therapeutic applications. Since the discovery of aptamers, many researchers have used aptamer selection as a means for generation of suitable binding partners for binding assay.

In various embodiments, determination of threshold levels of certain biomarkers can be indicative of a disease state in a subject. For example, markers of renal damage such as serum creatinine, creatinine clearance rates, cystatin C, and glomerular filtration rates, can be used for the prognosis and/or diagnosis of acute kidney injury.

In certain embodiments, in subjects with symptoms of acute heart failure (e.g., dyspnea) serum creatinine (sCr) was measured from blood samples drawn at various time intervals to assign an acute kidney injury (AKJ) status to each subject. For example, sCr levels were determined at time (T)=0,24,48,72, and 96 hours from admission (T=0 identical to admission). sCr also measured at discharge, and baseline (not admission) sCr level was determined. Differences between sCr at each draw and sCr at baseline were can be used to assess AKI status (i.e., the clinical endpoint of interest was AKJ status).

Acute kidney injury status can be assigned to the patients using different methods. For example, in one illustrative model, subjects with elevated sCr over two or more adjacent draws relative to baseline sCr, sCr(SSB), can be considered to be AKI positive.

In certain embodiments, a creatinine value for a draw at T, sCr(T), may be considered to be elevated if the ratio sCr(T)/sCr(SSB)≧2.5, preferably sCr(T)/sCr(SSB)≧2.0, and more preferably sCr(T)/sCr(SSB)≧1.5. In other embodiments, sCr(T), may be considered to be elevated if the ratio sCr(T)/sCr(SSB)≧1.4.1,3, 1,2, or 1.1.

In certain embodiments, a creatinine value for a draw at T, sCr(T), may be considered to be elevated if the difference sCr(T)−sCr(SSB)≧1.5 mg/dl, sCr(T)−sCr(SSB)≧1.0 mg/dl, sCr(T)−sCr(SSB)≧0.5 mg/dl, sCr(T)−sCr(SSB)≧0.4 mg/dl, sCr(T)−sCr(SSB)≧0.3 mg/dl, sCr(T)−sCr(SSB)≧0.2 mg/dl, or sCr(T)−sCr(SSB)≧0.1 mg/dl.

As used herein, the term “sustained” refers to an elevation of sCr that was elevated for two consecutive draws or more. In certain preferred embodiments, the elevation was considered to have occurred at the earliest T of the sustained elevation, For example, a patient who exhibited sCr elevations at T=0, 72, and 96 hours only would have been assigned AKI positive status at T=72 hours, but not at T=0. In certain illustrative embodiments, diseased (D) patients were considered to be those who were AKI positive at the admission (T=0) draw (i.e., admission and T=24 hour draws must have been elevated).

As used herein “non-diseased (ND) patients” were those who, taking into account missing draws as wed as those present, could not have had two or more consecutive elevated sCr values, For example, a patient whose admission through discharge draws were [N/A−+−+−] (+=elevated, −=non-elevated) would be assigned to be AKI negative (ND) because two consecutive elevated results could not be obtained regardless of the value of the admission draw. On the other hand, [N/A+−−−−] would be omitted because the presence consecutive elevated draws would depend on the status of the missing draw.

In another illustrative model (i.e., a transient model), subjects with an elevated sCr value at T were defined to be one for which sCr(T)−sCr(SSB)≧0.1 mg/dl, sCr(T) sCr(SSB)÷0.3 mg/dl, sCr(T)−sCr(SSB)≧0.4 mg/dl, sCr(T)−sCr(SSB)≧0.5 mg/dl, sCr(T)−sCr(SSB)≧0.6 mg/dl, sCr(T)−sCr(SSB)≧1.0 mg/dl, or sCr(T)−sCr(SSB)≧1.5 mg/dl. In certain embodiments, diseased patients were those who had an elevated sCr value at T=0, regardless of the remaining draws. For example, non-diseased subjects were those who were known to have non-elevated sCr at admission regardless of the remaining draws. In certain embodiments, this refers to a subject who bad elevated sCr values following the admission draw (but not at the admission draw) were defined to be ND. Patients with missing sCr(0) values were omitted.

Those skilled in the art will recognize that many approaches can be taken In producing antibodies or other binding partners, and screening and selecting for affinity and specificity for use in biomarker assays, but these approaches do not change the scope of the invention.

### EXAMPLE

The following examples serve to illustrate the present invention. These examples are in no way intended to limit the scope of the invention.

### Example 1

### Biochemical Analyses

Markers were measured using standard immunoassay techniques. These techniques involve the use of antibodies to specifically bind the analyte(s) of interest.

Immunoassays were performed using bead-based methods, or using microliter-based assays, or using microfluidtc devices manufactured at Biosite Incorporated essentially as described in WO98/43739, WO98/08606, WO98/21563, and WO93/24231.Analytes may be measured using a sandwich immunoassay or using a competitive immunoassay as appropriate, depending on the characteristics and concentration range of the analyte of interest.

Multiplexed and single-assay, bead-based immunoassays were performed on human plasma (or serum) samples in microliter plates. The primary antibody for each assay was conjugated to modified paramagnetic Lurninex® beads obtained from Radix Biosolutions. Either the secondary antibodies (sandwich assays) or the antigens (competitive assays) were hiotlnylated. Fluorescent signals were generated using Streptavidin-R-Phycoerythrin (SA-RPE: Prosymc PJ31S). All assays were heterogeneous and required multiple washes; washes were performed in 96-well plates placed on a 96-well magnetic ring stand (Ambion) in order to keep the paramagnetic beads from being removed. All liquid handling steps were performed with a Beckrnan Biomek FX.

An 8-point calibration curve was made gravimetrically by spiking each antigen into the calibration matrix. For sandwich assays, this matrix was plasma (or serum) from healthy donors; one of the eight points included free antibody to neutralize any endogenous antigen that was present. For competitive assays, this matrix was CDS buffer (10 mmol/L Tris-HCl (pH 8.0), 150 mmol/L NaCl, 1 mmol/L MgCl₂, 0.1 mmol/L ZnCl₂, 10 mL/L polyvinyl alcohol (MW 9000-10 000), 10 g/L bovine serum albumin, and 1 g/L NaN₃). Samples were stored in 384-well microliter plates kept at −70° C. A source plate was made by thawing the sample plate at 37° C., and then adding replicates of the 8-point calibration curve.

The assays were performed at room temperature. The bead-based primary antibody solution was added to a 384-well assay plate (10 μL/well) and then samples were added from the source plate (10 μl/well), mixed, and incubated one hour. Note, competitive assays were run in different assay plates than the sandwich assays, and the hiotlnylated antigen was added to the samples before transfer to the assay plate. Each 384-well plate was split into four 96-well plates for subsequent processing. The plates were washed as described above; the sandwich assays were incubated with biotinylated secondary antibodies and washed again. The assay mixtures were labeled with SA-RPE, washed, and mad using a Lumioex® LX200 reader; the median signal for each assay for was used for data reduction of each sample. The antigen concentrations were calculated using a standard curve determined by fitting a five parameter logistic function to the signals obtained for the 8-point calibration curves.

The assays were calibrated using purified proteins (that is either the same as or related to the selected analyte, and that can be detected in the assay) diluted gravimetrically into EDTA plasma treated in the same manner as the sample population specimens. Endogenous levels of the analyte present in the plasma prior to addition of the purified marker protein was measured and taken into account in assigning the marker values in the calibrators. When necessary to reduce endogenous levels in the calibrators, the endogenous analyte was stripped from the plasma using standard immunoaffinity methods. Calibrators were assayed in the same manner as the sample population specimens, and the resulting data used to construct a “dose-response” curve (assay signal as a function of analyte concentration), which may be used to determine analyte concentrations from assay signals obtained from subject specimens.

For a sandwich immunoassay in microtiter plates, a monoclonal antibody directed against a selected analyte was biotinylated using N-hydroxysuccinimide biotin NHSbiotin) at a ratio of about 5 NHS-biotin moieties per antibody. The antibody-biotin conjugate was then added to wells of a standard avium 384 well microliter plate, and antibody conjugate not bound to the plate was removed. This formed the “anti-marker” in the microtiter plate. Another monoclonal antibody directed against the same analyte was conjugated to alkaline phosphatase, for example using succinimidyl 4-N-[maleimidomethyl]-cyclohexane-1-carboxylate (SMCC) and N-succinimidyl 3-[2-pyridyldithio]propionate (SPDP) (Pierce, Rockford, Ill.).

Biotinylated antibodies were pipetted into mlcrotlter plate wells previously coated with avidin and incubated for 60 mm. The solution containing unbound antibody was removed, and the wells washed with a wash buffer, consisting of 20 mM borate (pH 7.42) containing 150 mM NaCl, 0,1% sodium, aside, and 0.02% Tween-20® (ICI Americas, Inc.). The plasma samples (10 μL) containing added HAMA inhibitors were pipeted into the microtiter plate wells, and Incubated for 60 min. The sample was then removed and the wells washed with a wash buffer. Use antibody-alkaline phosphatase conjugate was then added to the wells and Incubated for an additional 60 min, after which time, the antibody conjugate was removed and the wells washed with a wash buffer. A substrate, (AuoPhos4®, Promega, Madison, Wis.) was added to the wells, and the rate of formation of the fluorescent product is related to the concentration of the analyte in the sample tested,

Microfluidic devices used to perform assays were essentially as described in Chapter 41, entitled “Near Patient Tests: Triage® Cardiac System,” in The Immunoassay Handbook, 2nd ed., David Wild, ed., Nature Publishing Group, 2001.

For sandwich immunoassays, a plasma sample was added to the microfluidic device that contains all the necessary assay reagents, including human anti-mouse antibody (HAMA) inhibitors, in dried form. The plasma passed through a filter to remove particulate matter. Plasma entered a “reaction chamber” by capillary action. This reaction chamber contained fluorescent latex particle-antibody conjugates (hereafter called FETL-antibody conjugates) appropriate to an analyte of interest, and may contain FETL-antibody conjugates to several selected analyses. The FETL-antibody conjugates dissolved Into the plasma to form a reaction mixture, which was held in the reaction chamber for an incubation period (about a minute) to allow the analyte(s) of interest in the plasma to bind to the antibodies. After the incubation period, the reaction mixture moved down the detection lane by capillary action. Antibodies to the analyte(s) of interest were immobilized in discrete capture zones on the surface of a “detection lane.”

Analyte/antibody-FETL complexes formed in the reaction chamber were captured on an appropriate detection zone to form a sandwich complex, while unbound FETL-antibody conjugates were washed from the detection lane into a waste chamber by excess plasma.

The amount of analyte/antibody-FETL complex bound on a capture zone was quantified with a fluorometer (Triage® MeterPlus, Biosite Incorporated) and was related to the amount of the selected analyte in the plasma specimen.

### Example 2

### Use of Biomarkers Prognostically

The following study utilizes patents from the Coordinating Study Evaluating Outcomes of Advising and Counseling in Heart Failure (COACH) study, a multicenter, randomized, controlled trial in which 1023 patients were enrolled after hospitalization because of HF. See, Arch. Intern. Med. 168: 316-24, 2008. Patients were assigned to 1 of 3 groups: a control group (follow-up by a cardiologist) and 2 intervention groups with additional basic or intensive support by a nurse specializing in management of patients with HF. Patients were studied for 18 months. Primary end points were time to death or rehospitalization because of HF and the number of days lost to death or hospitalization.

A baseline WAP four-disulfide core domain protein 2 measurement was obtained from the COACH subjects. The baseline draw was taken after randomization to either the care or active Intervention pathway as described above, which was to have occurred within 2 days of HF admission. Descriptive statistics obtained from this measurement are presented in the following table. “N” is the number of subjects in each group; “25th”, “50th”, and “75th” refer to the value at the 25th”, 50th, and 75th percentile, respectively; “SD” is the standard deviation; SE of Mean is the standard error tor the mean value.

| | | | | | | DEATH, |
|---|---|---|---|---|---|---|
| | | | NO DEATH, | | | all cause |
| | | NO HF | NO HF | DEATH, | HF | OR HF |
| | NO DEATH | rehosp | rehosp | all cause | rehosp | rehosp |
| N | 479 | 419 | 327 | 92 | 148 | 240 |
| 0th percentile | 0.71 | 0.71 | 0.71 | 2.22 | 1.03 | 1.03 |
| 25th percentile | 3.18 | 3.15 | 2.87 | 4.87 | 4.06 | 4.41 |
| 50th percentile | 5.17 | 5.17 | 4.67 | 7.66 | 7.94 | 7.81 |
| 75th percentile | 9.26 | 8.69 | 7.66 | 17.17 | 12.29 | 13.93 |
| 100th percentile | 42.72 | 63.26 | 33.94 | 63.26 | 30.19 | 63.26 |
| Mean | 7.112 | 7.47 | 6.06 | 12.50 | 9.10 | 10.41 |
| SE | 0.262 | 0.35 | 0.27 | 1.16 | 0.51 | 0.55 |
| Variance | 32.60 | 51.93 | 22.96 | 123.49 | 38.75 | 73.60 |
| SD | 5.71 | 7.21 | 4.79 | 11.11 | 6.22 | 8.58 |

The ability of the baseline WAP four-disulfide core domain protein 2 measurement to identify outcome risk was determined. We computed adjusted odds ratios (AOR) for CVD and CHD death by marker level quartile, normalized to first quartile odds. For the fourth quartile, the AOR can be expressed as in the following equation:

$$AOR ( Q 4 ) = \frac{\frac{P ( + \vert Q 4 , X )}{P ( - \vert Q 4 , X )}}{\frac{P ( + \vert Q 1 , X )}{P ( - \vert Q 1 , X )}}$$

In the equation, P(+|Q4,X) is the probability of death, given that the subject's marker level fell within the fourth quartile, and that the value of the covariates to be adjusted for (e.g. age, gender) is X for all subjects used in the calculation. The numerator and denominator are the odds of death versus survival for the fourth and first quartiles respectively. We also used follow-up data on the clinical endpoints CVD and CHD death to compute empirical survival probabilities. We also modeled these data using Cox proportional hazards (CPH) regression, which allowed us to estimate the impact of marker level, age, gender, etc. on survival. Empirical estimates of the survival probability were computed using the Kaplan-Meier method, which accounts for censored data (i.e., subjects that exit the study due to causes other than the endpoint of interest). Appropriate methods which may be used for the analysis may be found in Dupont, William Dudley; Statistical modeling for biomedical researchers; a simple introduction to the analysis of complex data; Cambridge University Press; 2002; Collett, David; Modeling survival data in medical research; CRC Press; 2003; and Bender, Ralf, Augustin, Thomas and Blettner, Maria; Statistics in Medicine; 24; 1713; 2005.

TABLE 2

| | |
|---|---|
| Hazard Ratio (3^{rd} vs. | |
| 1^{st} tertiles) | P-Value |

Event: HF re-hospitalization or death (all cause)

| | | |
|---|---|---|
| WAP4C | 3.30 | 1.8E−12 |
| WAP4C, adjusted for COACH | 2.80 | 4.0E−08 |
| treatment group, age, gender, NYHA | | |
| class at enrollment | | |
| WAP4C, adjusted for COACH | 2.26 | 2.7E−05 |
| treatment group, age, gender, NYHA | | |
| class at enrollment, and BNP | | |
| WAP4C, adjusted for COACH | 2.65 | 1.6E−06 |
| treatment group, age, gender, | | |
| diabetes, LVEF, NYHA class at | | |
| enrollment | | |
| WAP4C, adjusted for COACH | 2.00 | 1.3E−03 |
| treatment group, age, gender, | | |
| diabetes, LVEF, NYHA class at | | |
| enrollment, and BNP | | |

Event: HF rehospitalization

| | | |
|---|---|---|
| WAP4C | 2.83 | 3.3E−07 |
| WAP4C, adjusted for COACH | 2.70 | 1.3E−05 |
| treatment group, age, gender, NYHA | | |
| class at enrollment | | |
| WAP4C, adjusted for COACH | 2.32 | 3.6E−04 |
| treatment group, age, gender, NYHA | | |
| class at enrollment, and BNP | | |
| WAP4C, adjusted for COACH | 2.79 | 2.3E−05 |
| treatment group, age, gender, | | |
| diabetes, LVEF, NYHA class at | | |
| enrollment | | |
| WAP4C, adjusted for COACH | 2.27 | 1.4E−03 |
| treatment group, age, gender, | | |
| diabetes, LVEF, NYHA class at | | |
| enrollment, and BNP | | |

TABLE 3

| | |
|---|---|
| Odds Ratio (3^{rd} vs. | |
| 1^{st} tertiles | P-Values |

Event: HF re-hospitalization or death (all cause)

| | | |
|---|---|---|
| WAP4C | 4.21 | <0.001 |
| WAP4C, adjusted for COACH | 3.26 | <0.001 |
| treatment group, age, gender, | | |
| WAP4C, adjusted for COACH | 2.69 | <0.001 |
| treatment group, age, gender, NYHA | | |
| class at enrollment, and BNP | | |

Event: HF rehospitalization

| | | |
|---|---|---|
| WAP4C | 2.47 | <0.001 |
| WAP4C, adjusted for COACH | 2.37 | 0.001 |
| treatment group, age, gender, | | |
| WAP4C, adjusted for COACH | 2.21 | 0.005 |
| treatment group, age, gender, NYHA | | |
| class at enrollment, and BNP | | |

| | AUC | | | | |
|---|---|---|---|---|---|
| | (confidence | | p- | N | N |
| Clinical Dichotomy | interval) | S.E. | Value | (control) | (disease |
| HF rehospitalization or death | 0.69 | 0.023 | <0.001 | 327 | 240 |
| (all cause) | (0.64-0.73) | | | | |
| HF rehospitalization or death | 0.61 | 0.032 | <0.001 | 321 | 101 |
| (all cause) (T > 180 days) | (0.55-0.68) | | | | |
| HF rehospitalization or death | 0.72 | 0.026 | <0.001 | 428 | 139 |
| (all cause) (T <= 180 days) | (0.67-0.77) | | | | |
| HF rehospitalization | 0.61 | 0.027 | <0.001 | 419 | 148 |
| | (0.56-0.66) | | | | |
| HF rehospitalization | 0.60 | | | | |
| (T > 180 days) | (0.52-0.67) | 0.037 | <0.005 | 353 | 69 |
| HF rehospitalization | 0.66 | | | | |
| (T <= 180 days) | (0.59-0.72) | 0.034 | <0.001 | 488 | 79 |

### Example 3

### CKD Progression

The following study utilizes patents from the Coordinating Study Evaluating Outcomes of Advising and Counseling in Heart Failure (COACH) study, a multicenter, randomized, controlled trial, in which 1023 patients were enrolled after hospitalization because of HF. See, Arch, Intern. Med. 168: 316-24, 2008. Patient samples were assayed to evaluate the utility of several biomarkers to aid in assigning an increased likelihood of CKD progression to a patient diagnosed with CKD. Samples obtained from each patient were analyzed by immunoassay to determine the level of each biomarker. Immunoassays were either operated in a sandwich assay format (for the determination of the markers Pentraxin 3, ANP propeptide, BNP, D-Dimer, ESAM, Galectin 3, GDF-15, LTBR, Mesothelin, MFO, Neuropilin 1, NGAL plasma specific, NTProCNP, Osteopontin, Periostin, PIGR, PSAP-B, RAGE, ST-2, Syndecan-I, TNFR1A, Troy, VEGFR1, WAP4C) or in a competitive assay format (for the determination of the markers Angiogenic, CRT, Cystatin C, NGAL, NRP-1) as described in more detail herein below.

Multiplexed bead-based immunoassays were performed on human, plasma (or serum) samples in microtiter plates. The primary antibody for each assay was conjugated to modified paramagnetic Luminex beads obtained from Radix Biosolutions. Either the secondary antibodies (sandwich assays) or the antigens (competitive assays) were biotinylated. Fluorescent signals were generated using Streptavidin-R-Phycoerythrin (SA-RPE: Prozyme PJ31S). All assays were heterogeneous and required multiple washes; washes were performed in 96-well plates placed on a 96-well magnetic ring stand (Ambion) in order to keep the paramagnetic beads from being removed. All liquid handling steps were performed with a Beckman Biornek FX.

An 8-point calibration curve was made gravimetrically by spiking each antigen into the calibration matrix. For sandwich assays, this matrix was plasma (or serum) from healthy donors; one of the eight points included free antibody to neutralize any endogenous antigen that was present. For competitive assays, this matrix was CDS buffer (10 mmol/L Tris-HCl (pH 8.0), 150 mmol/L NaCl, 1 mmol/L MgC12, 0.1 mmol/L ZnC12, 10 mL/L polyvinyl alcohol (MW 9000-10 000), 10 g/L bovine serum albumin, and 1 g/L NaN3). Samples were stored in 384-well microliter plates kept at −70° C. A source plate was made by thawing the sample plate at 37° C., and then adding replicates of the 8-point calibration curve.

The assays were performed at room temperature. The bead-based primary antibody solution was added to a 384-well assay plate (10 ul/well) and then samples were added from the source plate (10 ul/well), mixed, and incubated one hour. Note, competitive assays were run in different assay plates than the sandwich assays, and the biotinylated antigen was added to the samples before transfer to the assay plate. Each 384-well plate was split into four 96-well plates for subsequent processing. The plates were washed as described above; the sandwich assays were incubated with biotinylated secondary antibodies and washed again. The assay mixtures were labeled with SA-RPE, washed, and read using a Luminex LX200 reader; the median signal for each assay was used for data reduction of each sample. The antigen concentrations were calculated using a standard curve determined by filling a five parameter logistic function to the signals obtained for the 8-point calibration curves.

The assignment of CKD progression status to a subject followed two discrete methods. In a first method a subjects CKD stage assignment when discharged from hospital was recorded. Subjects had follow up visits after 6, 12, 18 months respectively from initial discharge. At each follow up visit a sample was collected from each subject and their CKD stage assignment was also determined. In each case, determination of CKD stage assignment was made based solely on estimated Glomerular Filtration Rate (eGFR) values (computed from serum creatinine measurements). Threshold eGFR values for the stages were taken from the CKD Executive Summary document (see American journal of Kidney Diseases, Vol 39, No 2, Suppl 1 (February) 2002, ppS17-S31). All subjects were CKD stage 3 when they were discharged from hospital Subjects with a missing eGFR value at any of the 4 sampling points (discharge, 6,12, or 18 months post discharge respectively) were omitted from the analysis.

Within the first method, two approaches fur classification of subjects at the four time points where samples were collected were used. In a first approach “positives” for CKD progression, i.e. those subjects whose CKD status had worsened during the period since discharge from hospital, were defined to be those subjects whose CKD stage at 6 and 12 months following discharge was 3, 4, or 5; in addition to having a CKD stage equal to 4 or 5 at 18 months post discharge. Conversely, any subject for which a sample had been obtained at each time point and which did not satisfy the set criteria tor CKD progression was considered as “negative”. In a second approach “positive” subjects were classified as in the first approach, with the exception that “negatives” were defined using a different approach. In this case a “negative”was defined as those subjects whose CKD stage at 6, 12, and 18 months after discharge from hospital was determined as 1,2, or 3.

In a second method of classifying subjects, again the use of “positive” and “negative” definitions was used. Two definitions of “negative” were used. Subjects categorized as “positives” for CKD progression were defined to be those patients whose eGFR at 6 and 12 months after discharge was lower feat their eGFR when they were discharged from hospital; in addition to having an eGFR at 18 months post discharge satisfying the conditions that either the eGFR at 18 months is less than half the eGFR at discharge OR the eGFR at discharge less the eGFR at 18 months is greater than 25 ml/min/1.73 m². Subjects with a missing eGFR value at any of the 4 draws (discharge, 6, 12, 18 months post discharge respectively) were omitted.

Subjects were categorized as “negative” when either (i) a sample had been obtained at each of the three follow up visits, but which did not meet the criteria for “positive”; or (ii) when eGFR at 6 months after discharge was greater than the eGFR at discharge AND when eGFR at 12 months after discharge was greater than eGFR at discharge, AND when eGFR at 18 months after discharge did not satisfy the relationship eGFR at 18 months is less than one half the eGFR at discharge OR eGFR at discharge less eGFR at 18 months is greater than 25 ml/min/1.73 m² (i.e. eGFR_{discharge}−eGFR_{18 months}>25 ml/min./l.73 m²).

Results of the analysis of subject data using the methods described above are presented in Tables 4, 5, 6 and 7. Comparing tables 4 and 5 shows that inclusion of the “negative” definitions when evaluating data resulted in slight Increases in the area under the curve for the ROC analysis of marker performance in the assignment of increased likelihood of future CKD progression to a subject.

Data presented in tables 6 and 7 show the outcome when analyzing subject samples using the second method of classifying subjects, when using only “positive” definitions (table 6) or “positive” and “negative” definitions (table 7). The data indicate the second method behaves quite differently to the first method, evidenced by the different ranking of markers, hut more noticeably by the markers that showed likelihood of identifying subject status in future.

Table 4 AUC data for analysis using first method with only “positive” definitions.

TABLE 4

| Marker | AUC | S.E. | pV-alue |
|---|---|---|---|
| TNFR1A | 0.801 | 0.55 | <0.001 |
| Troy | 0.75 | 0.061 | <0.001 |
| Galectin 3 | 0.73 | 0.059 | <0.001 |
| NTProCNP | 0.726 | 0.059 | <0.001 |
| ESAM | 0.708 | 0.071 | 0.002 |
| WAP4C | 0.703 | 0.063 | <0.001 |
| PIGR | 0.699 | 0.064 | <0.001 |
| NGAL | 0.696 | 0.065 | 0.001 |

Table 5 AUG data for analysis using first method with “positive” and “negative” definitions.

TABLE 5

| Marker | AUC | S.E. | p-Value |
|---|---|---|---|
| TNFR1A | 0.81 | 0.055 | <0.001 |
| Troy | 0.756 | 0.062 | <0.001 |
| NTProCNP | 0.734 | 0.059 | <0.001 |
| Galectin 3 | 0.728 | 0.059 | <0.001 |
| ESAM | 0.719 | 0.071 | 0.001 |
| NGAL | 0.717 | 0.065 | <0.001 |
| WAP4C | 0.709 | 0.062 | <0.001 |
| PIGR | 0.704 | 0.065 | <0.001 |

Table 6 AUG data for analysis using second method with only “positive” definitions.

TABLE 6

| Marker | AUC | S.E. | p-Value |
|---|---|---|---|
| RAGE | 0.667 | 0.061 | 0.003 |

Table AUC data for analysis using second method with “positive” and “negative” definitions.

TABLE 7

| Marker | AUC | S.E. | p-Value |
|---|---|---|---|
| Troy | 0.698 | 0.067 | 0.002 |
| NTProCNP | 0.666 | 0.08 | 0.019 |
| NGAL | 0.643 | 0.067 | 0.017 |

The data presented in Tables 4-7 indicate there is some variation in the area under curve, as well as the probability of an event occurring, for any given marker according to the model used for data analysis. For example, TNFR1A has the highest rank when analyzed using the first method, however, it has the lowest rank when analyzed using the second method. Whereas Troy has a similar rank in all methods of analysis. When method one is used, all samples had a probability p<0.05, indicating the statistical likelihood of the outcome for each marker. This was not the case for the second method in which case two markers where only “positive” definitions were used in the analysis bad p>0.05; thus demonstrating the improvement in outcome when both “positive” and “negative” definitions were utilized in processing subject sample data.

### Example 4

### Acute Kidney Injury

The following study was designed to assess the clinical utility of 3rd heart sound and investigate systolic and diastolic (left and right) ventricular function using echocardiographic methods.

Subjects were presented to the emergency department with symptoms of acute heart failure (e.g., dyspnea) Serum creatinine (sCr) measured from blood samples drawn at T=0, 24, 48, 72, and 96 hours from admission (T=0 identical to admission) sCr also measured at discharge, and baseline (not admission) sCr level was determined.

Differences between sCr at each draw and sCr at baseline were used to assign an acute kidney injury (AKI) status to each subject. The clinical endpoint of interest was AKI status.

Two methods were used to define AKI status:

Sustained: Above threshold sCr values in two (or more) consecutive draws. Threshold for sustained method: sCr(T)/sCr(baseline)≧1.5 or sCr(T)−sCr(baseline)≧0.5 mg/dl (T=any serial draw). Earlier of the consecutive above threshold draws defined to be the draw at which subject became AKI positive.

Transient: Above threshold sCr value in any single serial draw. Threshold for transient method: sCr(T)−sCr(baseline)>=0.3 mg/dl.

The analysis results shown below were designed to evaluate the clinical utility of 3rd heart sound and investigate systolic and diastolic ventricular function using echocardiographic methods. Study subjects presented to the emergency room with symptoms of acute HF. Serum creatinine (sCr) was measured from a series of blood draws scheduled to be taken at T=0, 24, 48, 72, and 96 hours from time of admission. sCr was also to be measured at discharge, and a steady state baseline level, sCr(SSB), (corresponding to pre-admission) was also determined.

Acute kidney injury status was assigned to the patients using two different methods. In the first scheme (sustained method), patients with elevated sCr over two or more adjacent draws relative to baseline sCr, sCr(SSB). were defined to be AKI positive. The creatinine value for a draw at T, sCr(T), was considered to be elevated if either the ratio sCr(T)/sCr(SSB)≧1.5 or the difference sCr(T)−sCr(SSB)≧0.5 mg/dl. An elevation of sCr was defined to be sustained if sCr was elevated for two consecutive draws or more. Furthermore, the elevation was considered to have occurred at the earliest T of the sustained elevation. For example, a patient who exhibited sCr elevations at T=0, 72, and 96 hours only would have been assigned AKI positive status at T=72 hours, but not at T=0. In the ROC table using the sustained definition, diseased (D) patients were considered to be those who were AKI positive at the admission (T=0) draw (i.e. admission and T=24 hour draws must have been elevated). In the same table, patients who became AKI positive at T>0 were omitted. Patients who had missing draws that made it impossible to determine whether there were two consecutive elevated sCr values were also omitted from the test. Non-diseased (ND) patients were those who, taking into account missing draws as well as those present, could not have had two or more consecutive elevated sCr values. For example, a patient whose admission through discharge draws were [N/A−+−+−] (+=elevated, −=non-elevated) would be assigned to be AKI negative (ND) because two consecutive elevated results could not be obtained regardless of the value of the admission draw. On the other hand, [N/A+−−−−] would be omitted because the presence consecutive elevated draws would depend on the status of the missing draw.

In the second method (transient), an elevated sCr value at T was defined to be one for which sCr(T)−sCr(SSB)≧0.3 mg/dl. In the ROC table using the transient definition, diseased patients were those who had an elevated sCr value at T=0, regardless of the remaining draws. Non-diseased subjects were those who were known to have non-elevated sCr at admission regardless of the remaining draws. This means that patients who bad elevated sCr values following the admission draw (but not at the admission draw) were defined to be ND, Patients with missing sCr(0) values were omitted.

Table 8 illustrates results of subjects with sustained AKI status.

| Marker | AUC | SE | p-Value | ND | D | LCI | UCI | Sense |
|---|---|---|---|---|---|---|---|---|
| WAP4C | 0.916 | 0.042 | <0.001 | 41 | 7 | 0.835 | 0.998 | 1 |
| sCr | 0.878 | 0.048 | <0.001 | 83 | 9 | 0.784 | 0.971 | 1 |

Table 9 illustrates results of subjects with transient AKI status.

| Marker | AUC | SE | p-Value | ND | D | LCI | UCI | Sense |
|---|---|---|---|---|---|---|---|---|
| WAP4C | 0.935 | 0.042 | <0.001 | 56 | 11 | 0.852 | 1.018 | 1 |
| sCr | 0.901 | 0.033 | <0.001 | 107 | 18 | 0.837 | 0.964 | 1 |

One skilled in the art will readily appreciate that the present invention is well adapted to carry out the objects and obtain the ends and advantages mentioned, as well as those inherent therein. The examples provided herein are representative of preferred embodiments, are exemplary, and are not intended as limitations on the scope of the invention.

It will be readily apparent to a person skilled in the art that varying substitutions and modifications may be made to the invention disclosed herein without departing from the scope and spirit of the invention.

All patents and publications mentioned in the specification are indicative of the levels of those of ordinary skill in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

Other embodiments are set forth within the following claims.

What is claimed is:

1. A method of assessing renal function in a subject suspected of having renal injury, comprising:
performing an assay that detects an amount of WAP four-disulfide cure domain protein 2 in a biological sample obtained from said subject; and
correlating the amount of WAP four-disulfide core domain protein 2 with the subject's renal function.

2. A method according to claim 1, wherein the correlating step comprises determining a concentration of WAP four-disulfide core domain protein 2 and relating said concentration to the occurrence or nonoccurrence of acute kidney injury in the subject.

3. A method according to claim 2, wherein the relating step comprises assigning an occurrence of acute kidney injury to the subject when said WAP four-disulfide core domain protein 2 concentration is greater than a predetermined threshold WAP four-disulfide core domain protein 2 concentration, or assigning a nonoccurrence of acute kidney injury to the subject when said WAP four-disulfide core domain protein 2 concentration is less than a predetermined WAP four-disulfide core domain protein 2 baseline concentration.

4. A method according to claim 3, wherein the predetermined WAP four-disulfide core domain protein 2 baseline concentration is determined by performing an assay method that detects WAP four-disulfide core domain protein 2 on a body fluid sample obtained from said subject at a time earlier than the time at which the body fluid sample used to provide the assay result was obtained.

5. A method according to claim 4, wherein the predetermined WAP four-disulfide core domain protein 2 baseline concentration has been determined from a first population of subjects suffering from acute kidney injury and a second population of subjects not suffering from acute kidney injury.

6. A method according to claim 5, wherein the predetermined WAP four-disulfide core domain protein 2 baseline concentration separates said first population from the second population with an odds ratio of at least 2 or more or 0.5 or less.

7. A method according to claim 5, wherein the predetermined WAP four-disulfide core domain protein 2 baseline concentration separates said first population from the second population with an odds ratio of at least 3 or more or 0.33 or less.

8. A method according to claim 5, wherein the predetermined WAP four-disulfide core domain protein 2 baseline concentration separates said first population from the second population with a specificity of at least about 70%.

9. A method according to claim 5, wherein the predetermined WAP four-disulfide core domain protein 2 baseline concentration separates said first population from the second population with a sensitivity of at least about 70%.

10. A method according to claim 1, wherein the biological sample is selected from the group consisting of urine, blood, serum, saliva, stool, and plasma.

11. A method according to claim 1, wherein the method further comprises:
determining one or more additional variables selected from the group consisting of a BMP level, an NT-proBNP level a proBNP level; a myeloperoxidase level, a soluble FLT-1 level, a C-reactive protein level, a cardiac troponin level, an NGAL level, a serum creatinine level, a creatinine clearance rate, a cystatin C level, and a glomerular filtration rate for said patient.

12. A method according to claim 1, wherein the method further comprises:
determining one or more additional variables selected from the group consisting of a urine output level for said subject, age of said subject, the presence or absence of diabetes in said subject, and the presence or absence of hypertension in said patient.

13. A method according to claim 1, wherein the threshold WAP four-disulfide core domain protein 2 concentration is between about 15 ng/mL and about 25 ng/mL.

14. A method according to claim 1, wherein the threshold WAP four-disulfide core domain protein 2 concentration is about 20.2 ng/mL.

15. A method according to claim 1, wherein the threshold WAP four-disulfide core domain protein 2 concentration is determined by detecting protein levels in said biological sample

16. The method of claim 14, wherein the protein levels are detecting using ELISA.

17. A method according to claim 1, wherein the threshold WAP four-disulfide core domain protein 2 concentration is determined by detecting mRNA encoding WAP four-disulfide core domain protein 2 in said biological sample.

18. The method of claim 17, wherein the mRNA is detected by RT-PCR.

19. A method of assessing renal function in a subject comprising:
a. obtaining a biological sample from said subject;
b. determining a concentration of WAP four-disulfide core domain protein 2 in the sample;
c. comparing the concentration of WAP four-disulfide core domain protein 2 in the sample to a threshold concentration of WAP four-disulfide core domain protein 2; and
d. determining if the subject is likely to have renal injury if the concentration of WAP four-disulfide core domain protein 2 in the sample is within a certain threshold concentration.

20 A. kit comprising:
reagents for performing an assay configured to detect WAP four-disulfide core domain protein 2; and
a device which contains an encoded calibration curve for correlating results from performing said assay to a concentration of WAP four-disulfide core domain protein 2,
wherein the concentration range of said calibration curve comprises a normal concentration of WAP four-disulfide core domain protein 2 and a threshold concentration of WAP four-disulfide core domain protein 2 used to diagnose acute kidney injury.
